(19) United States
(12) Patent Application Publication
(10) Pub. No.: US 2021/0023233 A1
(43) Pub. Date: Jan. 28, 2021

(54) ANTIBODY-PEPTIDE COMPLEXES AND USES THEREOF

(71) Applicant: Mayo Foundation for Medical Education and Research, Rochester, MN (US)

(72) Inventors: Wendy K. Nevala, Rochester, MN (US); Svetomir N. Markovic, Rochester, MN (US); John T. Butterfield, Edina, MN (US); Daniel J. Knauer, Costa Mesa, CA (US)

(73) Assignee: Mayo Foundation for Medical Education and Research, Rochester, MN (US)

(21) Appl. No.: 16/967,229

(22) PCT Filed: Feb. 6, 2019
(86) PCT No.: PCT/US19/16823

§ 371 Date: Aug. 4, 2020

Related U.S. Application Data
(60) Provisional application No. 62/626,799, filed on Feb. 6, 2018.

(51) Int. Cl. A61K 47/64; A61K 47/68; G01N 33/574; A61K 33/243

(52) U.S. Cl. CPC A61K 47/64; A61K 47/6803; A61K 33/243; G01N 33/57492; A61K 47/6851

(57) ABSTRACT

Materials and methods for diagnosing and treating clinical conditions such as cancer using polypeptide complexes that include (a) a polypeptide component, (b) a therapeutic agent or a label, and (c) an antibody, are provided herein. Specifically, the polypeptide complex comprising: (a) a specified polypeptide component, (b) one or more cisplatin molecules or the polypeptide is labeled with 99Tcm, and (c) a monoclonal antibody that specifically binds to a tumor antigen, for treating or diagnosing cancer, respectively.

### CROSS-REFERENCE TO RELATED APPLICATIONS

This application claims priority from U.S. Provisional Application Ser. No. 62/626,799, filed Feb. 6, 2018, the disclosure of which is incorporated herein by reference in its entirety.

### TECHNICAL FIELD

This document relates to methods and materials that can be used to, for example, detect cells (e.g., cancer cells) in vitro or within mammals (e.g., humans) for diagnosis of clinical conditions such cancer, as well as materials and methods for treating cancer. For example, this document provides polypeptide complexes that include (a) a polypeptide linked to (b) a therapeutic agent or a label and complexed with (c) an antibody, where the polypeptide complexes can be used as diagnostic and therapeutic tools.

### BACKGROUND

Chemotherapy remains a mainstay of systemic therapy for many types of cancer, including melanoma. Most chemotherapeutics are only slightly selective to tumor cells, and toxicity to healthy proliferating cells can be high (Allen, Cancer 2:750-763, 2002), often requiring dose reduction and even discontinuation of treatment. One way to overcome chemotherapy toxicity issues and improve drug efficacy is to target chemotherapy drugs to tumors using antibodies that are specific for proteins selectively expressed (or overexpressed) by tumor cells, thereby altering the biodistribution of the chemotherapy and resulting in more drug going to the tumor and less drug going to healthy tissue. Monoclonal antibodies (e.g., bevacizumab, rituximab, and trastuzumab) that have been developed to treat cancer can be used to inhibit the activity of a targeted polypeptide within a mammal being treated, or to deliver other agents (e.g., radiotherapeutic agents or toxic drugs) to cells expressing the targeted polypeptide.

Conventional antibody-dependent chemotherapy has been designed with a toxic agent linked to a targeting antibody via a synthetic protease-cleavable linker. For example, humanized therapeutic monoclonal antibodies, including bevacizumab, trastuzumab and rituximab, bind with a high affinity to albumin bound paclitaxel nanoparticles (ABRAXANE®), thereby providing the ability to specifically target the paclitaxel to tumors. The efficacy of such therapy typically is dependent on the ability of the antibody to bind to target cells, the ability of the linker to be cleaved, and the uptake of the toxic agent into the target cell (Schrama et al., Nature Reviews. Drug Discovery 5:147-159, 2006). Challenges to the effectiveness of antibody-targeted chemotherapy have included the instability of the linkers between the antibody and chemotherapy drug, reduced tumor toxicity of the chemotherapeutic agent when bound to the antibody, and the inability of the conjugate to bind and enter tumor cells.

### SUMMARY

This document is based, at least in part, on the development of polypeptide complexes that include (a) a polypeptide component, and (b) cisplatin, with or without an antibody. This document also is based, at least in part, on the development of polypeptide complexes that include (a) a technetium 99 (⁹⁹Tcm)-labeled polypeptide, and (b) an antibody. Such polypeptide complexes can be used as diagnostic and therapeutic tools, and may provide advantages over directly labeled or coupled antibodies that lack the polypeptide component.

As described herein, in some embodiments, a polypeptide-antibody complex can include a polypeptide component that contains the amino acid sequence VVLNQLCVLHEKTPVSDR (SEQ ID NO:1), or a variant of SEQ ID NO:1, where the polypeptide is labeled with ⁹⁹Tcm. In some embodiments, a polypeptide-antibody complex can include a polypeptide component that contains the sequence VVLCQLCVLHEKTPVSDR (SEQ ID NO:2, in which a Cys is substituted for the Asn at position 4 of SEQ ID NO:1), or a variant of SEQ ID NO:2, where one or more cisplatin molecules are coupled to the polypeptide via one or more of the Cys residues.

The ⁹⁹Tcm-labeled or cisplatin-coupled polypeptides can be complexed with various antibodies. To form a polypeptide-antibody complex as described herein, one or more antibodies (e.g., therapeutic antibodies) can be non-covalently attached to a polypeptide provided herein. Thus, the polypeptide components of the complexes provided herein can non-covalently bind to therapeutic monoclonal antibodies such as, without limitation, bevacizumab, rituximab, trastuzumab, alemtuzumab, atezolizumab, blinatumomab, brentuximab, cetuximab, denosumab, dinutuximab, ibritumomab, ipilimumab, nivolumab, obinutuzumab, ofatumumab, panitumumab, pembrolizumab, pertumumab, gemtuzumab, tositumomab, muromanab, and biosimilars thereof. The polypeptide-antibody complexes can be used to treat, for example, cancers in which tumors or cancer cells express a marker recognized by the antibody component of the complexes. The polypeptide-antibody complexes also can be used in vitro or in vivo to visualize cells or tissues expressing antigens targeted by the antibody of the complex. For example, a polypeptide-antibody complex provided herein can be used to image cells or tissue (e.g., tumor tissue) using clinical tests such as scintigraphy, positron-emission tomography (PET), and single-photon emission computed tomography (SPECT). This flexible tool may be employed to ascertain patient tumor burden in most, if not all, cancer types that have tumor-specific protein expression.

In one aspect, this document features a polypeptide complex containing (a) a polypeptide having the sequence set forth in SEQ ID NO:2, or a sequence having at least 88% identity to SEQ ID NO:2; (b) one or more cisplatin molecules; and (c) a monoclonal antibody that specifically binds to a tumor antigen. The polypeptide can contain a sequence having at least 94% identity to SEQ ID NO:2. The polypeptide can have the sequence of SEQ ID NO:2. The tumor antigen can be vascular endothelial growth factor (VEGF), programmed death-ligand 1 (PD-L1), HER2, or CD20.

In another aspect, this document features a method for treating a tumor in a mammal. The method can include administering to the mammal a polypeptide complex containing (a) a polypeptide having the sequence set forth in SEQ ID NO:2, or a sequence having at least 88% identity to SEQ ID NO:2; (b) one or more cisplatin molecules; and (c) a monoclonal antibody that specifically binds to a tumor antigen. The polypeptide can contain a sequence having at least 94% identity to SEQ ID NO:2. The polypeptide can have the sequence of SEQ ID NO:2. The polypeptide complex can be administered in an amount effective to reduce a symptom associated with the tumor in the mammal. The polypeptide complex can be administered in an amount effective to reduce the size of the tumor in the mammal. The tumor antigen can be VEGF, PD-L1, HER2, or CD20.

In addition, this document features the use of a polypeptide complex for treating a tumor in a mammal. The polypeptide complex includes (a) a polypeptide containing the sequence set forth in SEQ ID NO:2, or a sequence having at least 88% identity to SEQ ID NO:2; (b) one or more cisplatin molecules; and (c) a monoclonal antibody that specifically binds to a tumor antigen. The polypeptide can contain a sequence having at least 94% identity to SEQ ID NO:2. The polypeptide can have the sequence of SEQ ID NO:2. The tumor antigen can be VEGF, PD-L1, HER2, or CD20.

In another aspect, this document features the use of a polypeptide complex in the manufacture of a medicament for treating a tumor in a mammal. The polypeptide complex can include (a) a polypeptide containing the sequence set forth in SEQ ID NO:2, or a sequence having at least 88% identity to SEQ ID NO:2; (b) one or more cisplatin molecules; and (c) a monoclonal antibody that specifically binds to a tumor antigen. The polypeptide can contain a sequence having at least 94% identity to SEQ ID NO:2. The polypeptide can have the sequence of SEQ ID NO:2. The tumor antigen can be VEGF, PD-L1, HER2, or CD20.

In another aspect, this document features a polypeptide complex containing (a) a polypeptide having the sequence set forth in SEQ ID NO:1, or a sequence having at least 88% identity to SEQ ID NO:1, where the polypeptide is labeled with ⁹⁹Tcm; and (b) a monoclonal antibody that specifically binds to a tumor antigen. The polypeptide can contain a sequence with at least 94% identity to SEQ ID NO:1. The polypeptide can have the sequence of SEQ ID NO:1. The tumor antigen can be VEGF, PD-L1, HER2, or CD20.

This document also features a method for detecting a cell expressing a selected antigen. The method can include contacting the cell with a polypeptide complex that contains (a) a polypeptide having the sequence set forth in SEQ ID NO:1, or having a sequence with at least 88% identity to SEQ ID NO:1, where the polypeptide is labeled with ⁹⁹Tcm, and (b) a monoclonal antibody that specifically binds to the antigen; and detecting the ⁹⁹Tcm on the cell. The polypeptide can contain a sequence having at least 94% identity to SEQ ID NO:1. The polypeptide can have the sequence of SEQ ID NO:1. The antigen can be VEGF, PD-L1, HER2, or CD20. The contacting can be in vitro or in vivo. The cell can be within a mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are a pair of chromatography plots showing time dependent conjugation of antibody binding peptide (SEQ ID NO:2; “AP”) with cisplatin. A 5 molar excess of cisplatin was added to a 1 mg/ml solution of the peptide in saline. At the indicated time points, samples were separated and analyzed using Total Ion Chromatography LC-MS. The peptide alone eluted at about 6 minutes, while the cisplatin-conjugated peptide eluted at about 5.3 minutes (FIG. 1A). The primary complex of the conjugate peak corresponds to one peptide molecule (2050 Da) bound to 2 dechlorinated cisplatin molecules, for a total molecular weight of 2577 Da (FIG. 1B).

FIGS. 2A-2C are a series of plots showing that cisplatin binding to peptide (SEQ ID NO:2; “AP”) is increased with lower pH and higher molar ratio. Cisplatin was added to a 1 mg/ml peptide solution at a 1:1, 5:1, or 10:1 Cis:peptide molar ratio, as indicated. Solutions were unadjusted saline (FIG. 2A), saline pH 4.0 (FIG. 2B), or saline pH 9.0 (FIG. 2C). At multiple time points, the samples were separated and analyzed using Total Ion Chromatography LC-MS. The peptide alone eluted at about 6 minutes, while the cisplatin-conjugated peptide eluted at about 5.3 minutes.

FIG. 3 is a graph plotting the kinetics of peptide- (SEQ ID NO:2; “AP”) cisplatin conjugate binding to rituximab. Rituximab was immobilized onto a Biacore CMS chip using amine coupling. A peptide-cisplatin conjugate was purified using Total Ion Chromatography, lyophilized, and re-suspended in HBS running buffer. Peptide-cisplatin was run at a range of 0.6 μM to 20 μM (indicated by the different lines) over the immobilized rituximab, using Biacore x100 Surface Plasmon Resonance technology. 120 second association, 3000 second dissociation. Kinetics were analyzed using Biacore Evaluation Suite.

FIG. 4 is a graph plotting the toxicity of cisplatin-loaded peptide. CD20+ Daudi human lymphoma cells were treated overnight with 100 μg/ml ABRAXANE® (ABX), 100 μg/ml cisplatin (CIS), 1 mg/ml rituximab (RIT), 1 mg/ml peptide only (PEP), 100 μg/ml peptide-loaded cisplatin (PEP+CIS), peptide with rituximab (PEP+RIT), or 100 μg/ml peptide-loaded cisplatin with rituximab (PEP+CIS+RIT). These data show that the cisplatin-containing peptide diminished Daudi proliferation to 80% of the total proliferation and was equivalent to ABRAXANE® and cisplatin alone, suggesting cisplatin in the context of the peptide is equally as toxic. Further, the addition of the clinical antibody (rituximab) did not compromise cisplatin toxicity.

FIGS. 5A and 5B are elution charts showing conjugation of peptide (SEQ ID NO:1; “AP”) with technetium. Technetium in pertechnate form was reduced with CORM A1 to Tc(CO)₃(H₂O)₃, incubated with a molar excess of AP, and assayed by HPLC using UV Vis detection at 215 nm. The elution chart of FIG. 5A shows a primary peptide peak at 15.841 minutes. The elution chart of FIG. 5B shows a primary peptide peak at 15.240 minutes, as well as a secondary peak at 19.743 minutes that corresponds to a new peptide complex, most likely in the technetium-conjugated form since nothing else varied between the two runs.

### DETAILED DESCRIPTION

This document provides a polypeptide having the sequence VVLNQLCVLHEKTPVSDR (SEQ ID NO:1) that is radiolabeled with ⁹⁹Tcm and, in some cases, bound to one or more therapeutic monoclonal antibodies. In some embodiments, the polypeptide can include one or more variations within SEQ ID NO:1.

As described herein, a radiolabeled polypeptide containing the sequence set forth in SEQ ID NO:1, or a variant of SEQ ID NO:1, has the ability to bind to various antibodies (e.g., tumor specific antibodies such as bevacizumab, rituximab, trastuzumab, alemtuzumab, atezolizumab, blinatumomab, brentuximab, cetuximab, denosumab, dinutuximab, ibritumomab, ipilimumab, nivolumab, obinutuzumab, ofatumumab, panitumumab, pembrolizumab, pertumumab, gemtuzumab, tositumomab, and muromanab), forming a complex that in turn can bind to the cell membrane-bound ligand for the antibody, thereby labeling cells positively for the membrane-bound target. This provides a diagnostic tool consisting of a single reagent that can be used as a tool to determine tumor burden. For example, a polypeptide component provided herein can be ⁹⁹Tcm labeled and noncovalently bound to a tumor specific-antibody, which then can be used in clinical testing to evaluate patient tumor burden in cancers that have tumor-specific protein expression (e.g., breast cancer, melanoma, lymphoma, renal cell carcinoma, ovarian cancer, lung cancer, and head and neck cancer). The labelled polypeptide also provides a research tool that can be used in the development of cancer therapies, for example.

This document also provides a polypeptide having the sequence VVLCQLCVLHEKTPVSDR (SEQ ID NO:2), which can be coupled to cisplatin and bound to multiple therapeutic monoclonal antibodies. In some embodiments, the polypeptide can include one or more variations within SEQ ID NO:2, including variations to substitute Cys for one or more amino acid residues within SEQ ID NO:2, and variations to substitute Ile for one or more amino acid residues within SEQ ID NO:2 (e.g., a Leu→Ile substitution at position 9 of SEQ ID NO:2, as set forth in SEQ ID NO:3).

As described herein, a cisplatin-coupled polypeptide containing the sequence set forth in SEQ ID NO:2, or a variant of SEQ ID NO:2, has the ability to bind to various antibodies (e.g., tumor specific antibodies such as bevacizumab, rituximab, trastuzumab, alemtuzumab, atezolizumab, blinatumomab, brentuximab, cetuximab, denosumab, dinutuximab, ibritumomab, ipilimumab, nivolumab, obinutuzumab, ofatumumab, panitumumab, pembrolizumab, pertumumab, gemtuzumab, tositumomab, and muromanab), forming a complex that in turn can bind to the cell membrane-bound ligand for the antibody, thereby delivering the cisplatin to the cell that express the membrane-bound target. This provides a therapeutic agent that can be used to treat various types of cancers (e.g., breast cancer, melanoma, lymphoma, renal cell carcinoma, ovarian cancer, lung cancer, and head and neck cancer).

The terms “purified” and “isolated” as used herein with reference to a polypeptide mean that the polypeptide (1) is not associated with proteins found in nature, (2) is free of other proteins from the same source, (3) is expressed by a cell from a different species, or (4) does not occur in nature. An isolated or purified polypeptide can be, for example, encoded by DNA or RNA, including synthetic DNA or RNA, or some combination thereof.

The term “substantially pure,” with reference to a polypeptide, means the polypeptide is substantially free of other polypeptides, lipids, carbohydrates, and nucleic acid with which it is naturally associated. A substantially pure polypeptide can be any polypeptide that is removed from its natural environment and is at least 60 percent pure. A substantially pure polypeptide can be at least about 65, 70, 75, 80, 85, 90, 95, or 99 percent pure, or about 65 to 75, 75 to 80, 80 to 85, 85 to 90, 90 to 95, or 95 to 99 percent pure. Typically, a substantially pure polypeptide will yield a single major band on a non-reducing polyacrylamide gel. In some embodiments, a substantially pure polypeptide can be a chemically synthesized polypeptide.

Any method can be used to obtain a substantially pure polypeptide. For example, polypeptide purification techniques, such as affinity chromatography and HPLC, as well as polypeptide synthesis techniques can be used. In addition, any material can be used as a source to obtain a substantially pure polypeptide. For example, tissue from wild-type or transgenic animals can be used as a source material. In addition, tissue culture cells engineered to over-express a particular polypeptide can be used to obtain substantially pure polypeptide. Further, a polypeptide can be engineered to contain an amino acid sequence that allows the polypeptide to be captured onto an affinity matrix. For example, a tag such as c-myc, hemagglutinin, polyhistidine, or FLAG™ tag (Kodak) can be used to aid polypeptide purification. Such tags can be inserted anywhere within the polypeptide, including at either the carboxyl terminus or the amino terminus, or in between. Other fusions that can be used include enzymes that aid in the detection of the polypeptide, such as alkaline phosphatase.

In some embodiments, a polypeptide as provided herein can include one or more variants as compared to the sequence set forth in SEQ ID NO:1 or SEQ ID NO:2. For example, the polypeptides provided herein can contain the amino acid sequence set forth in SEQ ID NO:1 or SEQ ID NO:2, except that the amino acid sequence can contain from one to three (e.g., one to three, two to three, one to two, three, two, or one) amino acid additions, subtractions, and/or substitutions, or modifications. In some embodiments, for example, a polypeptide can contain the amino acid sequence set forth in SEQ ID NO:1 or SEQ ID NO:2 with one to three amino acid residue substitutions (e.g., substitutions to replace the original amino acid residue with a Cys) that allow for conjugation to cisplatin as described herein.

In some embodiments, the amino acid change(s) do not substantially reduce the ability of the polypeptide to bind to a therapeutic antibody. As used herein, the phrase “retaining antibody specificity” indicates that the antibodies associated with the polypeptides provided herein continue to recognize (bind to) the epitope being targeted. For example, variations within a polypeptide sequence as set forth herein can be present so long as such modifications do not substantially reduce the ability of the polypeptide to bind an antibody. As described below, for example, conservative substitutions may be made that do not substantially reduce binding affinity.

Any amino acid residue set forth in the sequences provided herein can be subtracted, and any amino acid residue (e.g., any of the 20 conventional amino acid residues or any other type of amino acid such as ornithine or citrulline) can be added to or substituted within the sequences set forth herein. The majority of naturally occurring amino acids are L-amino acids, and naturally occurring polypeptides are largely comprised of L-amino acids. D-amino acids are the enantiomers of L-amino acids. In some cases, a polypeptide as provided herein can contain one or more D-amino acids.

Polypeptides having one or more amino acid additions, subtractions, or substitutions relative to SEQ ID NO:1 or SEQ ID NO:2 (also referred to herein as “variant” polypeptides) can be prepared and modified as described herein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Substitutions between isosteric amino acid residues (e.g., glutamic acid and glutamine, or aspartic acid and asparagine) also may be considered to be conservative.

Further examples of conservative substitutions that can be made at any position within the polypeptides provided herein are set forth in TABLE 1.

TABLE 1
Examples of conservative amino acid substitutions

| Original Residue | Exemplary substitutions |
|---|---|
| Ala | Val, Leu, Ile |
| Arg | Lys, Gln, Asn |
| Asn | Gln, His, Lys, Arg |
| Asp | Glu |
| Cys | Ser |
| Gln | Asn |
| Glu | Asp |
| Gly | Pro |
| His | Arg, Asn, Gln, Lys |
| Ile | Leu, Val, Met, Ala, Phe, Norleucine |
| Leu | Ile, Norleucine, Val, Met, Ala, Phe |
| Lys | Arg, Gln, Asn |
| Met | Leu, Phe, Ile |
| Phe | Leu, Val, Ile, Ala |
| Pro | Gly |
| Ser | Thr |
| Thr | Ser |
| Trp | Tyr |
| Tyr | Phe, Trp, Thr, Ser |
| Val | Leu, Ile, Met, Phe, Ala, Norleucine |

In some embodiments, a polypeptide can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be assessed by determining the ability of the peptide variant to bind to an antibody using, for example, methods disclosed herein.

In some embodiments, a polypeptide as provided herein can have a length of 15 to 21 amino acid residues (e.g., 15 to 18, 16 to 19, 17 to 20, 18 to 21, 15 to 17, 16 to 18, 17 to 19, 18 to 20, 19 to 21, 15 to 16, 16 to 17, 17 to 18, 18 to 19, 19 to 20, 20 to 21, 15, 16, 17, 18, 19, 20, or 21 amino acid residues).

In some embodiments, a polypeptide can have the amino acid sequence set forth in SEQ ID NO:1 or SEQ ID NO:2, but with a particular number of amino acid substitutions. For example, a polypeptide can have the amino acid sequence of SEQ ID NO:1 or SEQ ID NO:2, but with one, two, or three amino acid substitutions.

In some embodiments, a polypeptide as provided herein can include an amino acid sequence with at least 83% (e.g., at least 88%, or at least 94%, or 100%) sequence identity to SEQ ID NO:1 or SEQ ID NO:2. Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm.BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:1), or by an articulated length (e.g., 20 consecutive nucleotides or amino acid residues from a sequence set forth in an identified sequence), followed by multiplying the resulting value by 100. For example, an amino acid sequence that has 16 matches when aligned with the sequence set forth in SEQ ID NO:1 is 88.9 percent identical to the sequence set forth in SEQ ID NO:1 (i.e., 16±18×100=88.9). It is noted that the percent sequence identity value typically is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Isolated polypeptides can be produced using any suitable method, including solid phase synthesis, and can be generated using manual techniques or automated techniques (e.g., using an Applied BioSystems (Foster City, Calif.) Peptide Synthesizer or a Biosearch Inc. (San Rafael, Calif.) automatic peptide synthesizer). The polypeptides provided herein also can be produced recombinantly, or obtained commercially.

Variant polypeptides having conservative and/or non-conservative substitutions with respect to SEQ ID NO:1 or SEQ ID NO:2 can be screened for binding to antibodies using any of a number of assays. For example, the binding activity of a polypeptide can be evaluated in vitro using surface plasmon resonance (e.g., using a Biacore X-100 Surface Plasmon Resonance device (GE Healthcare; Chicago, Ill.).

This document also provides nucleic acid molecules encoding the polypeptides provided herein. For example, this document provides nucleic acid molecules encoding the polypeptides of SEQ ID NO:1 and SEQ ID NO:2, as well as nucleic acid molecules encoding polypeptides that are variants of the polypeptides having the amino acid sequences set forth in SEQ ID NO:1 and SEQ ID NO:2. Thus, a nucleic acid molecule as provided herein can encode a polypeptide that contains the amino acid sequence set forth in SEQ ID NO:1 or SEQ ID NO:2, except that the amino acid sequence contains one to three (e.g., one to three, two to three, one to two, three, two, or one) amino acid additions, subtractions, and substitutions as described herein.

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

Isolated nucleic acid molecules can be produced using standard techniques, including, without limitation, common molecular cloning and chemical nucleic acid synthesis techniques. For example, polymerase chain reaction (PCR) techniques can be used to obtain an isolated nucleic acid containing nucleotide sequence that encodes a polypeptide as provided herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

In some embodiments, the polypeptides provided herein are labeled with ⁹⁹Tcm, which can allow for detection of the polypeptide and other molecules with which the polypeptide associates. Methods for attaching ⁹⁹Tcm to a polypeptide include, without limitation, the method described in Example 2 herein, which includes reduced to Tc(CO)₃(H2O)₃ using sodium boranocarbonate (CORM A1) before mixing with the peptide.

In some embodiments, the polypeptides provided herein are coupled to one or more cisplatin molecules. In some cases, for example, a polypeptide as provided herein (e.g., SEQ ID NO:2) is associated with cisplatin before being combined with an antibody. Such polypeptide-cisplatin complexes can contain one, two, three, or four cisplatin molecules per polypeptide sequence. Methods for attaching cisplatin to a polypeptide include, without limitation, the method described the Examples 1 herein. As indicated in Examples 1 and 2, cisplatin coupling to the peptide is dependent on time, pH, and molar ratio, such that attachment increases as pH decreases, time increases, and molar ratio increases.

Polypeptides labeled with ⁹⁹Tcm or coupled to cisplatin as described herein can interact with various antibodies, including therapeutic monoclonal antibodies that bind to tumor antigens. Thus, this document also provides complexes that contain a polypeptide and an antibody.

The polypeptide component of a complex can have the amino acid sequence of SEQ ID NO:1 or SEQ ID NO:2, or the amino acid sequence of SEQ ID NO:1 or SEQ ID NO:2 with one to three additions, subtractions, or substitutions. The antibody component of a complex can be a monoclonal antibody targeted to a tumor antigen. As described herein, useful antibodies include, without limitation, bevacizumab, rituximab, trastuzumab, and atezolizumab. Methods for forming polypeptide-antibody complexes include those described in the known in the art, for example. In some cases, the polypeptide and the antibody of interest can be combined at a particular ratio (e.g., a v:v ratio). For example, when the polypeptide is at a 10 mg/ml concentration, an antibody:polypeptide ratio of about 1:10, 1:15, 1:20, 1:25, or 1:30 may be particularly useful.

The antibodies in the complexes provided herein can bind to particular tumor antigens. For example, the antibodies can bind to, without limitation, alphafetoprotein (AFP), carcinoembryonic antigen (CEA), CA-125, MUC-1, epithelial tumor antigen (ETA), melanoma-associated antigen (MAGE), tyrosinase, HER.2, HER3, CD3, CD19, CD20, CD33, CD47, CD274, CD279, CD30, CD52, PD-1, PD-L1, CTLA4, GD2, VEGF, BCR-ABL, NY-ESO-1, MAGE-1, MAGE-3, SSX2, Melan-A, EGFR, CD38, or RANK ligand.

Methods for making complexes containing a ⁹⁹Tcm-labeled or cisplatin-linked polypeptide and an antibody as described herein include those known in the art, such as those described in PCT Application No. PCT/US2017/045643, which is incorporated herein by -reference in its entirety. For example, a method for making a polypeptide-antibody complex can include incubating a ⁹⁹Tcm-labeled or cisplatin-coupled polypeptide with an antibody. The polypeptide can have the sequence set forth in SEQ ID NO:1 or SEQ ID NO:2, or the sequence set forth in SEQ ID NO:1 or SEQ ID NO:2 with one to three amino acid substitutions, additions, or subtractions. In some embodiments, complexes as provided herein can be formed by contacting a ⁹⁹Tcm-labeled or cisplatin-coupled polypeptide with an antibody at a ratio of about 10:1 to about 10:30 polypeptide:antibody. Exemplary ratios include, without limitation, about 10:2 to about 10:25, about 10:2 to about 1:1, about 10:2 to about 10:6, or about 10:4.

In general, any suitable combination of polypeptide and antibody can be used as described herein. For example, an appropriate amount of a ⁹⁹Tcm-labeled or cisplatin-coupled polypeptide and an appropriate amount of an antibody can be mixed together and incubated at a suitable temperature (e.g., room temperature, about 5° C. to about 60° C., about 23° C. to about 60° C., about 15° C. to about 30° C., about 15° C. to about 25° C., about 20° C. to about 30° C., or about 20° C. to about 25° C.) for a period of time (e.g., about 30 minutes, or from about 5 minutes to about 60 minutes, about 5 minutes to about 45 minutes, about 15 minutes to about 60 minutes, about 15 minutes to about 45 minutes, about 20 minutes to about 400 minutes, or about 25 minutes to about 35 minutes) before being administered to a patient having a cancer.

In general, the complexes provided herein can be formulated for administration to a patient by any accepted mode, including oral administration, systemic (e.g., transdermal, intranasal, or by suppository) administration, or parenteral (e.g., intramuscular, intravenous, or subcutaneous) administration. Various formulations and drug delivery systems are known in the art. See, e.g., Gennaro, ed. (1995) Remington's Pharmaceutical Sciences, 18th ed., Mack Publishing Co.

In some cases, the complexes and compositions provided herein can be formulated for direct injection into a tumor. Direct injection includes injection into or proximal to a tumor site, perfusion into a tumor, and the like.

The ⁹⁹Tcm-labeled and cisplatin-coupled polypeptide-antibody complexes described herein can be incorporated into compositions for use as research or clinical (e.g., diagnostic or therapeutic) tools. The compositions can include a polypeptide having the amino acid sequence of SEQ ID NO:1 or SEQ ID NO:2, or the amino acid sequence of SEQ ID NO:1 or SEQ ID NO:2 with one to three additions, subtractions, or substitutions, where the polypeptide is ⁹⁹Tcm-labeled or cisplatin-coupled as described herein and complexed with an antibody (e.g., a monoclonal therapeutic antibody), where the complex is combined with a carrier (e.g., water, saline, a suitable buffer, or another pharmaceutically acceptable carrier).

Pharmaceutically acceptable carriers include, for example, pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more labeled polypeptide-antibody complexes provided herein. In addition to water and saline, typical pharmaceutically acceptable carriers include, without limitation, binding agents (e.g., polyvinylpyrrolidone or hydroxypropyl methylcellulose), fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

In some cases, complexes containing a ⁹⁹Tcm-labeled or cisplatin-coupled polypeptide and an antibody can be stored prior to being administered to a patient. For example, a composition can be generated as described herein and stored for a period of time (e.g., days or weeks) prior to being administered to a patient. In some cases, lyophilized compositions can be prepared using standard lyophilization techniques, with or without the presence of stabilizers or buffers. Surprisingly, such conditions do not alter the relatively fragile structure of the peptide-antibody complexes. Thus, the lyophilized complexes, upon reconstitution in an aqueous solution, can retain the ability to recognize and bind to antigens in vivo.

Pharmaceutical compositions containing complexes described herein can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous drip), oral, topical (e.g., transdermal, sublingual, ophthalmic, or intranasal), or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

The polypeptides and polypeptide-antibody complexes, and compositions containing the polypeptides and polypeptide-antibody complexes described herein also can be used as research and diagnostic tools. Thus, this document also provides methods for using the polypeptides described herein to, for example, identify antibodies to which the polypeptides can bind and form complexes. In addition, this document provides methods for using a labeled polypeptide-antibody complex as described herein to detect cells that express a particular antigen (e.g., a tumor marker such as, without limitation, VEGF, PD-L1, HER2, CD20, alphafetoprotein (AFP), carcinoembryonic antigen (CEA), CA-125, MUC-1, epithelial tumor antigen (ETA), melanoma-associated antigen (MAGE), tyrosinase, HER3, CD3, CD19, CD33, CD47, CD274, CD279, CD30, CD52, PD-1, CTLA4, GD2, BCR-ABL, NY-ESO-1, MAGE-1, MAGE-3, SSX2, Melan-A, EGFR, CD38, or RANK ligand). The methods can include, for example, contacting a cell (e.g., a tumor cell, either in vitro or in vivo) with a complex that includes a ⁹⁹Tcm labeled polypeptide having the sequence of SEQ ID NO:1, or a variant of SEQ ID NO:1 having at least 83% (e.g., at least 88%, or at least 94%) identity to the sequence of SEQ ID NO:1, where the polypeptide is bound by an antibody that can bind selectively to the antigen. After incubation under conditions in which the antibody can interact with an antigen on the cell, if the antigen is present, the cells can be washed to remove unbound polypeptide-antibody complexes, and then evaluated to determine whether the presence of the label is detected on the cell. In vitro methods may include detection by autoradiography, for example, or immunofluorescent immunohistochemistry. When conducted in vivo, the methods can include detecting labeled cells via scintigraphy, PET, or SPECT, for example, or via other suitable techniques for assessing cancer patients.

The complexes and compositions containing cisplatin-coupled polypeptides combined with tumor-targeted antibodies, as described herein, can be useful in treating cancer cells and tumors in a mammal (e.g., a human). The term “treating” or “treatment” covers the treatment of a disease or disorder (e.g., cancer), in a subject, such as a human, and includes: (i) inhibiting a disease or disorder, i.e., arresting its development; (ii) relieving a disease or disorder, e.g., causing regression of the disease or disorder; (iii) slowing progression of the disease or disorder; and/or (iv) inhibiting, relieving, or slowing progression of one or more symptoms of the disease or disorder.

Thus, this document provides methods for treating a cancer cell by contacting the cell with an effective amount of a complex that contains an immunotherapeutic agent (an antibody) as described herein. An “effective amount” refers to an amount of an agent, complex, or composition that results in a reduction in proliferation of cancer cells, killing of cancer cells, a reduction in tumor size and/or tumor burden, prevention of metastasis, and the like. In some embodiments, the effective amount of cisplatin in a dose of a complex as provided herein can be about 20 mg/m² to about 150 mg/m² (e.g., about 20 to 30 mg/m², about 30 to 40 mg/m², about 40 to 50 mg/m², about 50 to 60 mg/m², about 60 to 70 mg/m², about 70 to 80 mg/m², about 80 to 90 mg/m², about 90 to 100 mg/m², about 100 to 110 mg/m², about 110 to 120 mg/m², about 120 to 130 mg/m², about 130 to 140 mg/m², about 140 to 150 mg/m², about 20 to 50 mg/m², about 50 to 100 mg/m², or about 100 to 150 mg/m²).

Cancers and tumors that can be treated using the polypeptide-antibody complexes, compositions, and methods described herein include, without limitation, biliary tract cancer, brain cancer (e.g., glioblastoma or medulloblastoma), breast cancer, uterine cancer, tubal cancer, cervical cancer, choriocarcinoma, colon cancer, bladder cancer, endometrial cancer, vaginal cancer, vulvar cancer, esophageal cancer, mouth cancer, gastric cancer, kidney cancer, hematological neoplasms (e.g., acute lymphocytic and myelogenous leukemia), multiple myeloma, AIDS-associated leukemias and adult T-cell leukemia, lymphoma, intraepithelial neoplasms (e.g., Bowen's disease and Paget's disease), liver cancer (hepatocarcinoma), lung cancer, head or neck cancers, oral cancers (e.g., cancers of the mouth, throat, esophageal, nasopharyngeal, jaw, tonsil, nasal, lip, salivary gland, or tongue), lymphomas (e.g., Hodgkin's disease and lymphocytic lymphomas), neuroblastomas, neuroendocrine tumors, including squamous cell carcinoma, adrenal cancer, anal cancer, angiosarcoma, appendix cancer, bile duct cancer, bone cancer, carcinoid tumors, soft tissue sarcoma, rhabdomyosarcoma, eye cancer, ovarian cancer (e.g., cancers arising from epithelial cells, stromal cells, germ cells, and mesenchymal cells), fallopian tube cancer, gall bladder cancer, pancreatic cancer, prostate cancer, rectal cancer, sarcomas (e.g., leiomyosarcoma, rhabdomyosarcoma, liposarcoma, fibrosarcoma and osteosarcoma), skin cancer (e.g., melanoma, Kaposi's sarcoma, basocellular cancer, and squamous cell cancer), testicular cancer (e.g., germinal tumors such as seminoma or non-seminoma teratomas and choriocarcinomas), stromal tumors and germ cell tumors, penile cancer, hemangioendothelioma, gastrointestinal cancer, ureteral cancer, urethral cancer, spinal cancer, pituitary gland cancer, primary central nervous system (CNS) lymphoma, thyroid cancer (e.g., thyroid adenocarcinoma and medullar carcinoma), and renal cancer (e.g., adenocarcinoma and Wilms tumor). In some embodiments, for example, the cancers and tumors that can be treated as described herein include breast cancer, prostate cancer, colorectal cancer, lymphoma, multiple myeloma, and melanoma.

Before administering a composition containing a complex as provided herein to a mammal, the mammal can be assessed to determine whether or not the mammal has a cancer or disease expressing the relevant antigen. Any suitable method can be used to determine whether or not a mammal has a cancer or disease expressing the relevant antigen. For example, a mammal (e.g., human) can be identified using standard diagnostic techniques. In some cases, a tissue biopsy can be collected and analyzed to determine whether or not a mammal has a cancer or disease expressing the antigen.

After identifying a mammal as having the disease or cancer, the mammal can be administered a composition containing a complex as provided herein. For example, a composition containing the complex can be administered prior to or in lieu of surgical resection of a tumor. In some cases, a composition containing a complex as provided herein can be administered following resection of a tumor.

If a particular mammal fails to respond to a particular amount, then the amount can be increased by, for example, two-fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the cancer or disease may require an increase or decrease in the actual effective amount administered.

A composition containing a complex as provided herein can be administered to a mammal in any appropriate amount, at any appropriate frequency, and for any appropriate duration effective to achieve a desired outcome (e.g., to increase progression-free survival). In some cases, a composition as provided herein can be administered to a mammal having a cancer or disease to reduce the progression rate of the cancer or disease by 5, 10, 25, 50, 75, 100, or more percent. For example, the progression rate can be reduced such that no additional cancer progression is detected.

Any suitable method can be used to determine whether or not the progression rate of cancer is reduced. For example, the progression rate of a cancer can be assessed by imaging tissue at different time points and determining the amount of cancer cells present. The amounts of cancer cells determined within tissue at different times can be compared to determine the progression rate. After treatment as described herein, the progression rate can be assessed again over another time interval. In some cases, the stage of cancer after treatment can be determined and compared to the stage before treatment to determine whether or not the progression rate was reduced.

In some cases, a composition as provided herein can be administered to a mammal having a cancer under conditions where progression-free survival is increased (e.g., by 5, 10, 25, 50, 75, 100, or more percent) as compared to the median progression-free survival of corresponding mammals having untreated cancer or the median progression-free survival of corresponding mammals having cancer treated with the polypeptide, chemotherapy agent, and antibody without forming complexes prior to administration. In some cases, a composition as provided herein can be administered to a mammal having a cancer to increase progression-free survival by 5, 10, 25, 50, 75, 100, or more percent as compared to the median progression-free survival of corresponding mammals having a cancer and having received the polypeptide, cisplatin, polypeptide-cisplatin without an antibody, or antibody alone. Progression-free survival can be measured over any length of time (e.g., one month, two months, three months, four months, five months, six months, or longer).

An effective amount of a composition containing complexes as provided herein can be any amount that reduces the progression rate of a cancer or disease expressing the antigen recognized by the binding agent, increases the progression-free survival rate, or increases the median time to progression without producing significant toxicity to the mammal. If a particular mammal fails to respond to a particular amount, then the amount can be increased by, for example, two-fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment, Various factors can influence the actual effective amount used for a particular application. For example, the frequency of administration, duration of treatment, use of multiple treatment agents, route of administration, and severity of the cancer or disease may require an increase or decrease in the actual effective amount administered.

The frequency of administration can be any frequency that reduces the progression rate of a cancer or disease, increases the progression-free or overall survival rate, or increases the median time to progression, without producing significant toxicity to the mammal. For example, the frequency of administration can be from about once a month to about three times a month, or from about twice a month to about six times a month, or from about once every two months to about three times every two months. The frequency of administration can remain constant or can be variable during the duration of treatment. A course of treatment with a composition as provided herein can include rest periods. For example, the composition can be administered over a two week period followed by a two week rest period, and such a regimen can be repeated multiple times. As with the effective amount, various factors can influence the actual frequency of administration used for a particular application, For example, the effective amount, duration of treatment, use of multiple treatment agents, route of administration, and severity of the cancer or disease may require an increase or decrease in administration frequency.

An effective duration for administering a composition provided herein can be any duration that reduces the progression rate of a cancer or disease, increases the progression-free survival rate, or increases the median time to progression without producing significant toxicity to the mammal. Thus, the effective duration can vary from several days to several weeks, months, or years. In general, the effective duration for the treatment of a cancer or disease can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the cancer or disease.

After administering a composition provided herein to a mammal, the mammal can be monitored to determine whether or not the cancer or disease was treated. For example, a mammal can be assessed after treatment to determine whether or not the progression rate of the cancer or disease was reduced. As described herein, any suitable method can be used to assess progression and survival rates.

In some cases, a ⁹⁹Tcm-labeled polypeptide-antibody complex can be used as a tool to aid in diagnosis. For example, a melanoma metastasis in the brain might not express the antigen (e.g., PD-L1) expressed by the primary tumor, such that while the primary tumor might respond well to anti-PD-L1 immunotherapy, the brain tumor would not respond due to lack of the antigen. Thus, one or more labeled polypeptide-antibody complexes can be used to ascertain what marker(s) are expressed by the secondary tumor. Armed with such information, a clinician could treat primary and secondary lesions effectively, using a combination of therapeutic agents. In addition, in some cases, a ⁹⁹Tcm-labeled polypeptide-antibody complex can be used as a tool to aid in monitoring the effectiveness of treatments as described herein.

Also provided herein are articles of manufacture containing one or more ⁹⁹Tcm-labeled polypeptides, cisplatin-coupled polypeptides, polypeptide-antibody complexes, or compositions as described herein. The components of an article of manufacture (e.g., the polypeptide(s) and the antibody(ies), or the polypeptide-antibody complex(es), and/or one or more buffers or diluents) can be provided separately or in combination, in one or more suitable containers. In some embodiments, the kit components (e.g., a combination of labeled polypeptide-antibody complexes) can be packaged as an in vitro diagnostic.

The invention will be further described in the following examples, which do not limit the scope of the invention described in the claims.

### EXAMPLES

### Example 1—Time Dependent Conjugation of AP with Cisplatin

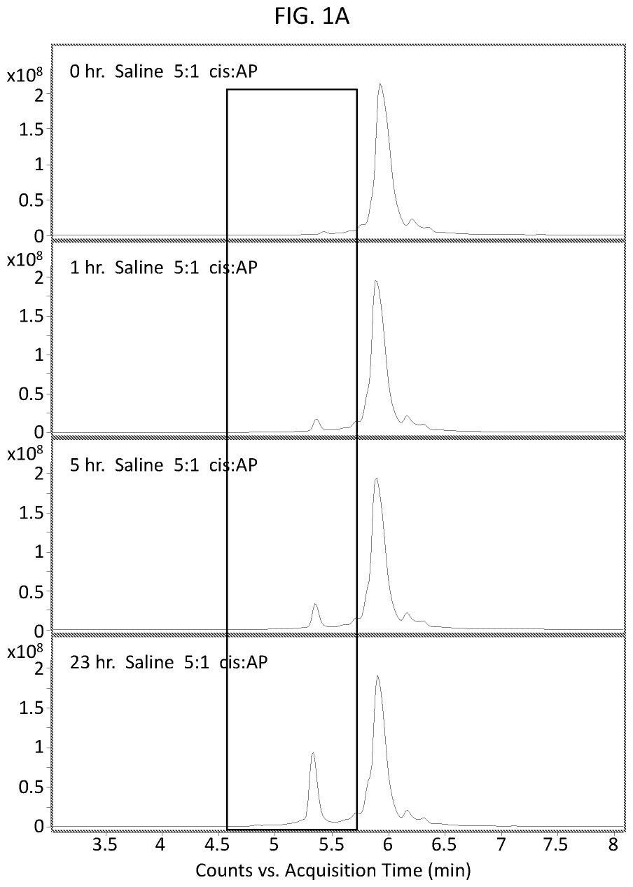

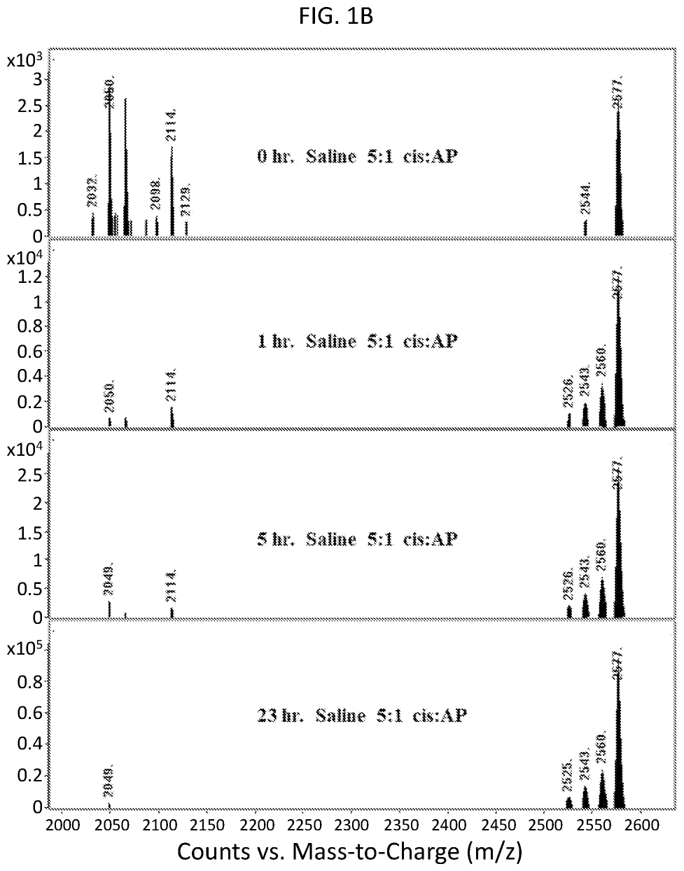

A 5 molar excess of cisplatin was added to a 1 mg/ml solution of SEQ ID NO:2 in saline. At 0 hours, 1 hour, 5 hours, and 23 hours, samples were separated and analyzed using Total Ion Chromatography LC-MS. The peptide alone eluted at about 6 minutes, while the cisplatin-conjugated peptide eluted at about 5.3 minutes (FIG. 1A). The primary complex of the conjugate peak corresponds to one peptide molecule (2050 Da) bound to two dechlorinated cisplatin molecules, for a total molecular weight of 2577 Da (FIG. 1B).

### Example 2—Cisplatin Binding to Peptide Increases with Lower pH and Higher Molar Ratio

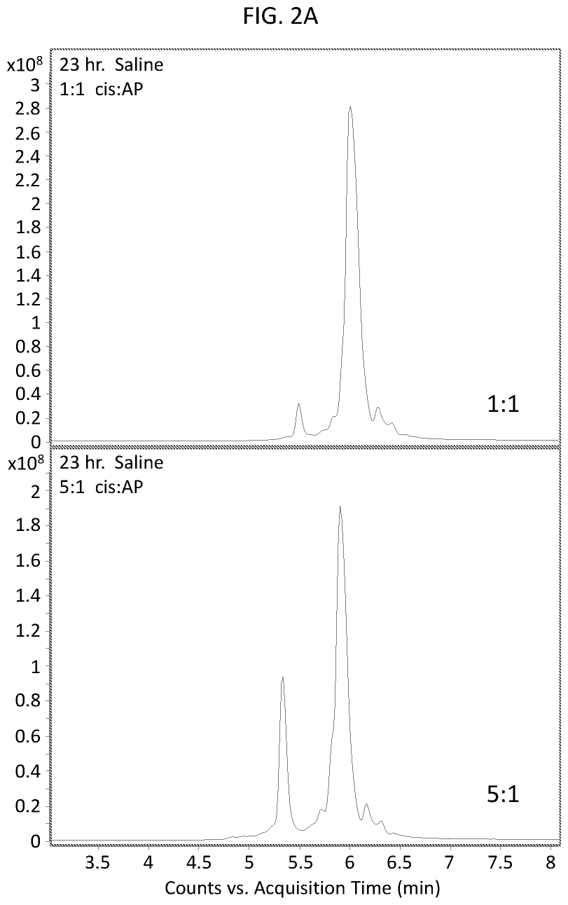

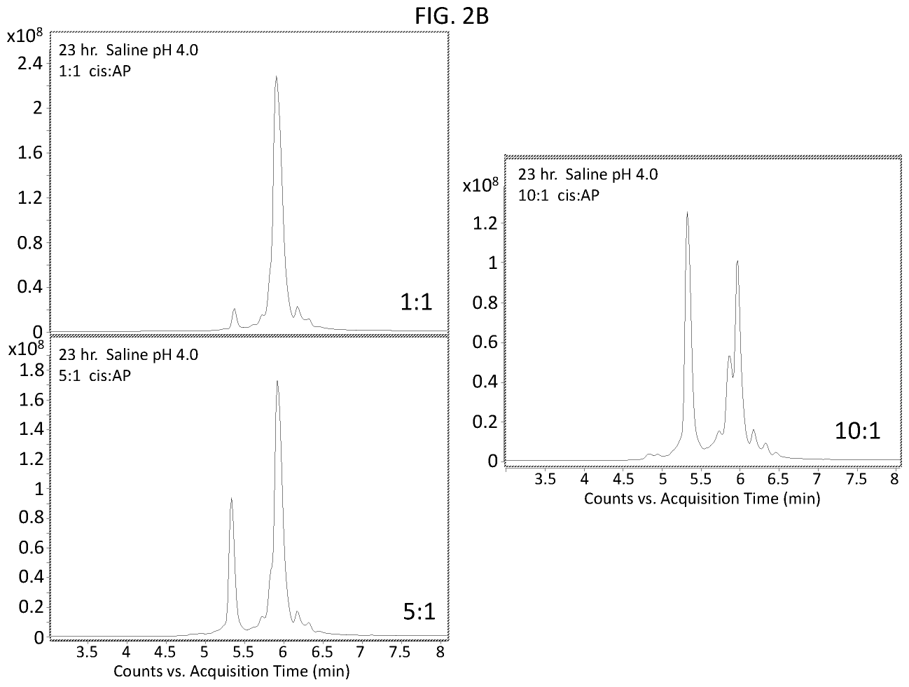

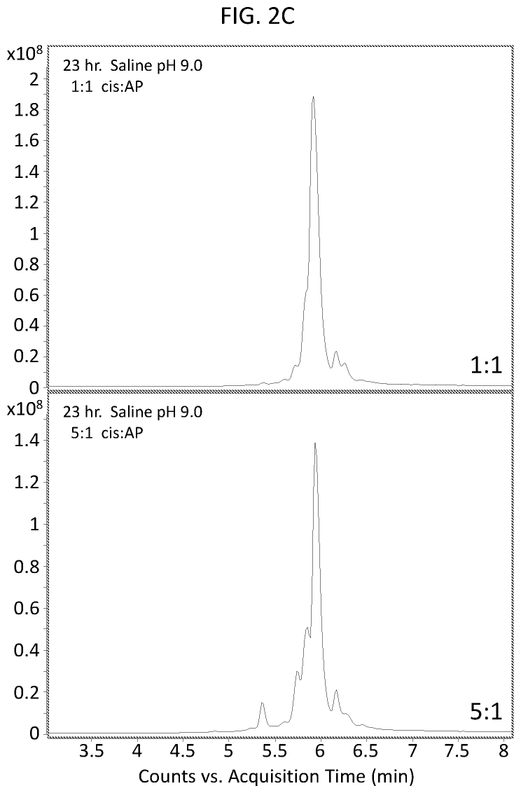

Cisplatin was added to a 1 mg/ml peptide (SEQ ID NO:2) solution at a 1:1, 5:1, or 10:1 Cis:peptide molar ratio. Solutions included unadjusted saline (FIG. 2A), saline at pH 4.0 (FIG. 2B), and saline at pH 9.0 (FIG. 2C). After 23 hours, the samples were separated and analyzed using Total Ion Chromatography LC-MS. The peptide alone eluted at about 6 minutes, while the cisplatin-conjugated peptide eluted at about 5.3 minutes.

### Example 3—Kinetics of Peptide-Cisplatin Binding to Rituximab

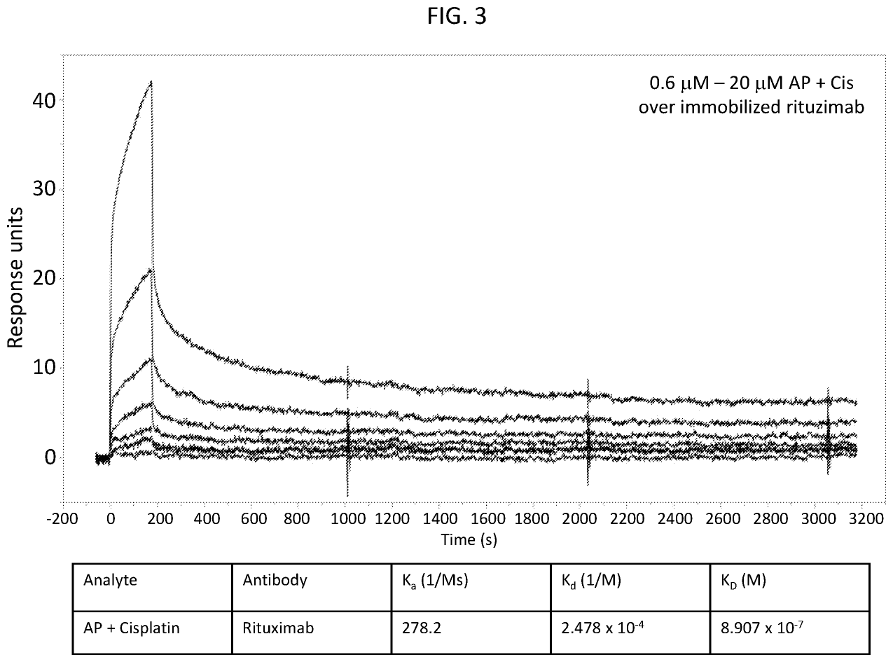

Rituximab was immobilized onto a Biacore CMS chip using amine coupling. A peptide (SEQ ID NO:2)-cisplatin conjugate was purified using Total Ion Chromatography, lyophilized, and re-suspended in HBS running buffer. Peptide-cisplatin was run over the immobilized rituximab at a range of 0.6 μM to 20 using Biacore x100 Surface Plasmon Resonance technology. Kinetics were analyzed using Biacore Evaluation Suite (FIG. 3).

### Example 4—Toxicity of Cisplatin-Loaded Peptide

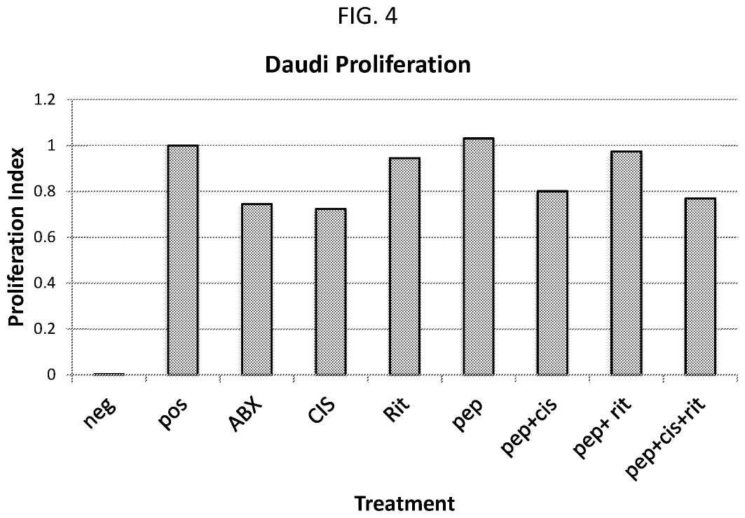

CD20+ Daudi human lymphoma cells were treated overnight with 100 μg/ml ABRAXANE® (ABX), 100 μg/ml cisplatin (CIS), 1 mg/ml rituximab (RIT), 1 mg/ml peptide (SEQ ID NO:2) only (PEP), 100 μg/ml peptide-loaded cisplatin (PEP+CIS), peptide with rituximab (PEP+RIT), or 100 μg/ml peptide-loaded cisplatin with rituximab (PEP+CIS+RIT), and cell survival was assessed (FIG. 4). The cisplatin-containing peptide diminished Daudi proliferation to 80% of the total (control) proliferation and was equivalent to ABRAXANE® and cisplatin alone, suggesting that cisplatin in the context of the peptide is equally toxic. Further, addition of rituximab did not compromise cisplatin toxicity.

### Example 5—Conjugation of Peptide to Technetium

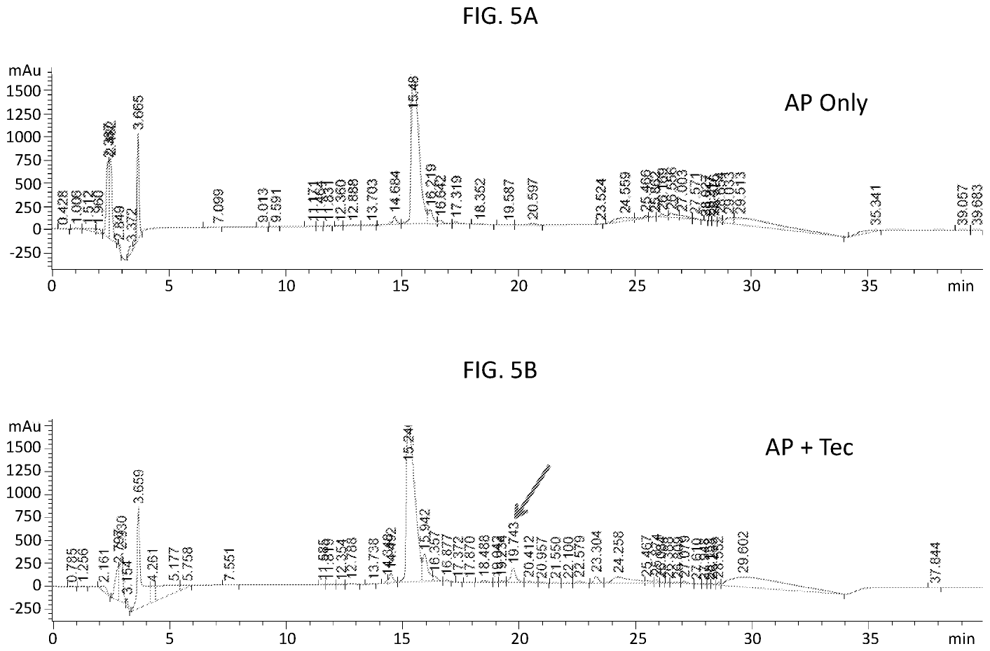

Technetium in pertechnate form was reduced with CORM A1 to Tc(CO)₃(H₂O)₃, incubated with a molar excess of peptide (SEQ ID NO:1), and assayed by HPLC using UV Vis detection at 215 nm. The peptide eluted at 15.841 minutes without the presence of technetium (FIG. 5A). The elution profile for peptide incubated with technetium showed a primary peptide peak at 15.240 minutes, as well as a secondary peak at 19.743 minutes (FIG. 5B) that corresponds to a new peptide complex, most likely in the technetium-conjugated form.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide complex comprising:
(a) a polypeptide comprising the sequence set forth in SEQ ID NO:2, or a sequence having at least 88% identity to SEQ ID NO:2;
(b) one or more cisplatin molecules; and
(c) a monoclonal antibody that specifically binds to a tumor antigen.

2. The polypeptide complex of claim 1, wherein the polypeptide comprises a sequence having at least 94% identity to SEQ ID NO:2.

3. The polypeptide complex of claim 1, wherein the polypeptide comprises the sequence of SEQ ID NO:2.

4. The polypeptide complex of claim 1, wherein the tumor antigen is vascular endothelial growth factor (VEGF), programmed death-ligand 1 (PD-L1), HER2, or CD20.

5. A method for treating a tumor in a mammal, the method comprising administering to the mammal a polypeptide complex, wherein said polypeptide complex comprises (a) a polypeptide comprising the sequence set forth in SEQ ID NO:2, or a sequence having at least 88% identity to SEQ ID NO:2; (b) one or more cisplatin molecules; and (c) a monoclonal antibody that specifically binds to a tumor antigen.

6. The method of claim 5, wherein the polypeptide comprises a sequence having at least 94% identity to SEQ ID NO:2.

7. The method of claim 5, wherein the polypeptide comprises the sequence of SEQ ID NO:2.

8. The method of claim 5, wherein the tumor antigen is VEGF, PD-L1, HER2, or CD20.

9-12. (canceled)

13. A polypeptide complex comprising:
(a) a polypeptide comprising the sequence set forth in SEQ ID NO:1, or a sequence having at least 88% identity to SEQ ID NO:1, wherein the polypeptide is labeled with ⁹⁹Tcm; and
(b) a monoclonal antibody that specifically binds to a tumor antigen.

14. The polypeptide complex of claim 13, wherein the polypeptide comprises a sequence having at least 94% identity to SEQ ID NO:1.

15. The polypeptide complex of claim 13, wherein the polypeptide comprises the sequence of SEQ ID NO:1.

16. The polypeptide complex of claim 13, wherein the tumor antigen is VEGF, PD-L1, HER2, or CD20.

17-20. (canceled)


SEQUENCE LISTING (3 sequences)

SEQ ID NO: 1 - protein, 18 aa, Homo sapiens
VVLNQLCVLHEKTPVSDR

SEQ ID NO: 2 - protein, 18 aa, artificial
  synthetic polypeptide
VVLCQLCVLHEKTPVSDR

SEQ ID NO: 3 - protein, 18 aa, artificial
  synthetic polypeptide
VVLCQLCVIHEKTPVSDR